(19) United States
(12) Patent Application Publication
(10) Pub. No.: US 2026/0359944 A1
(43) Pub. Date: Nov. 12, 2026

(54) BALANCED-IMAGE FILTERING IN MOTION FIELD ESTIMATION

(71) Applicant: Canon Kabushiki Kaisha, Tokyo (JP)

(72) Inventors: Yu-Jung TSAI, Vernon Hills, IL (US); Wenyuan QI, Vernon Hills, IL (US); Jeffrey KOLTHAMMER, Vernon Hills, IL (US)

(73) Assignee: Canon Kabushiki Kaisha, Tokyo (JP)

(21) Appl. No.: 19/644,853

(22) Filed: Apr. 10, 2026

Related U.S. Application Data
(60) Provisional application No. 63/801,371, filed on May 7, 2025.

(51) Int. Cl. G06T 5/60; A61B 6/00

(52) U.S. Cl. CPC G06T 5/60; A61B 6/5264; G06T 2207/10104; G06T 2207/10108

(57) ABSTRACT

A method for performing motion correction and motion compensation includes: receiving list-mode emission data acquired by using a medical imaging system to scan an object; acquiring a gating signal indicating gate times for the list-mode emission data; sorting, based on the gating signal, the list-mode emission data to obtain a plurality of gated emission datasets; reconstructing a plurality of gated images based on the plurality of gated emission datasets; determining a plurality of denoising control parameters based on noise information associated with the plurality of gated images; denoising, based on the plurality of denoising control parameters, the plurality of gated images to generate a plurality of denoised gated images; estimating, based on the plurality of denoised gated images, a plurality of motion vector fields for the plurality of gated images; and generating a motion-corrected image, based on the plurality of motion vector fields and the plurality of gated images.

### CROSS-REFERENCE TO RELATED APPLICATIONS

The present application claims the benefit of priority to provisional Application No. 63/801,371, filed on May 7, 2025, the entire contents of which are incorporated herein by reference.

### FIELD

This disclosure relates to medical imaging and, more particularly, to an apparatus and method for motion estimation and motion correction in imaging systems such as positron emission tomography (PET) scanners and single-photon emission computed tomography (SPECT) scanners.

### BACKGROUND

The background description provided herein is for the purpose of generally presenting the context of the disclosure. Work of the presently named inventors, to the extent the work is described in this background section, as well as aspects of the description that may not otherwise qualify as prior art at the time of filing, are neither expressly nor impliedly admitted as prior art against the present disclosure.

Medical imaging systems, such as positron emission tomography (PET) scanners and single-photon emission computed tomography (SPECT) scanners, are widely used for diagnosis and clinical interventions. To reduce image degradation caused by physiological motion (e.g., respiration), a known approach in the PET imaging modality is to estimate a three-dimensional (3D) motion vector field from a time-series (cine) of PET images, and then use the estimated motion vector field to transform the PET images from individual gates to a selected position within a breathing cycle. After the transformation, the cine or gated images can be combined to generate a final image with minimum motion while containing all available information.

Motion estimation in this workflow can be performed using an optical-flow-based method or similar methods that operate on the time-series input images. However, these methods can be sensitive to noise in the input images. In order to speed up the imaging workflow and/or reduce the injected dose, the cine images often have higher noise, which can adversely affect motion estimation and subsequent motion correction.

Accordingly, it is desirable to develop improved approaches for estimating motion vector fields and performing motion correction, particularly in scenarios in which the reconstructed cine images exhibit relatively high noise.

### SUMMARY

The present disclosure relates to a method for performing motion estimation and correction in a medical imaging system. The method includes: receiving list-mode emission data acquired by using the medical imaging system to scan an imaging object; acquiring a gating signal indicating gate times for the list-mode emission data; sorting, based on the gating signal, the list-mode emission data into a plurality of gates to obtain a plurality of gated emission datasets; reconstructing a plurality of gated images based on the plurality of gated emission datasets, wherein each gated image of the plurality of gated images is reconstructed from a respective one of the plurality of gated emission datasets; determining a plurality of denoising control parameters based on noise information associated with the plurality of gated images, wherein each denoising control parameter of the plurality of denoising control parameters corresponds to a respective one of the plurality of gated images; denoising, based on the plurality of denoising control parameters, the plurality of gated images to generate a plurality of denoised gated images, thereby balancing noise levels among the plurality of denoised gated images; estimating, based on the plurality of denoised gated images, a plurality of motion vector fields for the plurality of gated images, wherein each motion vector field of the plurality of motion vector fields corresponds to a respective one of the plurality of gated images; and generating a motion-corrected image of the imaging object, based on the plurality of motion vector fields and the plurality of gated images.

The disclosure additionally relates to an apparatus for performing motion estimation and correction in a medical imaging system. The apparatus includes processing circuitry configured to: receive list-mode emission data acquired by using the medical imaging system to scan an imaging object, acquire a gating signal indicating gate times for the list-mode emission data, sort, based on the gating signal, the list-mode emission data into a plurality of gates to obtain a plurality of gated emission datasets, reconstruct a plurality of gated images based on the plurality of gated emission datasets, wherein each gated image of the plurality of gated images is reconstructed from a respective one of the plurality of gated emission datasets, determine a plurality of denoising control parameters based on noise information associated with the plurality of gated images, wherein each denoising control parameter of the plurality of denoising control parameters corresponds to a respective one of the plurality of gated images, denoise, based on the plurality of denoising control parameters, the plurality of gated images to generate a plurality of denoised gated images, thereby balancing noise levels among the plurality of denoised gated images, estimate, based on the plurality of denoised gated images, a plurality of motion vector fields for the plurality of gated images, wherein each motion vector field of the plurality of motion vector fields corresponds to a respective one of the plurality of gated images, and generate a motion-corrected image of the imaging object, based on the plurality of motion vector fields and the plurality of gated images.

Note that this summary section does not specify every embodiment and/or incrementally novel aspect of the present disclosure or claimed invention. Instead, the summary only provides a preliminary discussion of different embodiments and corresponding points of novelty. For additional details and/or possible perspectives of the invention and embodiments, the reader is directed to the Detailed Description section and corresponding figures of the present disclosure as further discussed below.

### BRIEF DESCRIPTION OF THE DRAWINGS

Various embodiments of this disclosure that are proposed as examples will be described in detail with reference to the following figures, wherein like numerals reference like elements, and wherein:

FIG. 1 is a block diagram illustrating an exemplary workflow of motion estimation and motion correction, in accordance with embodiments of the present disclosure;

FIG. 2 shows a comparison of motion-compensated images generated from N (=5) gated images using different denoising schemes before optical-flow-based motion estimation, including normal denoising applied to the gated images and balanced denoising applied to the gated images;

FIG. 3 shows (a) an exemplary structure of a denoising deep convolutional neural network (DCNN), and (b) an exemplary weighting scheme for a weighted loss function used to train the denoising DCNN, in accordance with embodiments of the present disclosure;

FIG. 4 shows a flow chart of an exemplary procedure for performing motion estimation and motion correction, in accordance with embodiments of the present disclosure;

FIG. 5A is an illustration of a perspective view of a positron emission tomography (PET) scanner apparatus, in accordance with embodiments of the present disclosure; and

FIG. 5B is a schematic of a PET scanner apparatus and associated hardware, in accordance with embodiments of the present disclosure.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting.

For example, the order of discussion of the different steps as described herein has been presented for the sake of clarity. In general, these steps can be performed in any suitable order. Additionally, although each of the different features, techniques, configurations, etc. herein may be discussed in different places of this disclosure, it is intended that each of the concepts can be executed independently of each other or in combination with each other. Accordingly, the present invention can be embodied and viewed in many different ways.

Furthermore, as used herein, the words “a,” “an,” and the like generally carry a meaning of “one or more,” unless stated otherwise.

During positron emission tomography (PET) acquisition, respiration and cardiac motion can induce degradation of image quality and quantitative accuracy by causing deficiency of count and blurring of anatomical structures. Accordingly, motion correction is necessary to improve quantitative evaluation and to prevent decline in image quality during the PET acquisition. In order to minimize these repercussions, various motion estimation methods based on external motion measurements have been introduced. These methods include, for example, detection of pressure variations using pressure sensors, optical motion tracking systems, etc.

Reconstructed image frames can be obtained from dynamic acquisitions, gated acquisitions, or acquisitions framed based on a motion threshold. Registration of the reconstructed frames is a common and useful data-driven method to estimate relatively discrete (e.g., inter-frame) motion. Non-rigid registration has been applied to obtain cardiac and respiratory motion vector fields. A variety of motion models can be used, including affine models, B-spline models, higher-dimensional fields from optical flow, etc. However, data-driven alignment of the reconstructed frames may be limited by image statistics and possible redistribution of the radiotracer.

In low-count frames, registration convergence and accuracy can be improved by noise regularisation and by incorporating additional constraints from priors and spatio-temporal cost functions, physical models, and joint estimation of motion and other parameters. Even so, the high noise in low-count frames remains a practical challenge for image-based motion estimation.

The present disclosure provides an improved approach for performing motion estimation and motion correction in the presence of image noise, including scenarios in which the reconstructed frames (e.g., the gated images) are relatively noisy due to low-count imaging conditions.

As discussed previously, optical-flow-based motion estimation methods, and similar image-based motion estimation methods, commonly operate on reconstructed gated images. In a typical workflow, the acquired list-mode data is sorted into a plurality of gates, a set of gated images is reconstructed, and the gated images are provided to a motion estimation algorithm that estimates a motion vector field from the gated images. A particular challenge in such workflows is that the gated images are often noisy. As a result, motion estimation algorithms may latch onto noise and thereby output inaccurate motion vector field estimates.

Various approaches have been proposed to address this problem. For instance, these approaches may preprocess the input gated images before feeding them into the optical flow algorithm (or other motion estimation algorithms), so as to use denoised gated images in motion estimation. Denoising using Gaussian filters or trained neural networks is one such approach.

However, in practice, the gated images can have different noise levels. Such differences may occur across different patients and across different gates for a same patient, for example. If denoising is applied in a fixed or “blind” manner (e.g., applying a same denoising processing to an entire set of gated images), the resulting denoised images may still exhibit different noise levels.

Estimating a motion vector field between two gated images with substantially different noise levels can be challenging. For example, a structure that is treated as noise in one gated image may be treated as a tumor or a lesion in another gated image, and thus the perceived texture may differ across these images, which can make it difficult for the motion estimation algorithm to identify corresponding structures and accurately estimate the motion vector field.

To address the above problem, the present disclosure provides an improved motion estimation and correction approach based on gated images. The approach includes performing noise analysis on the gated images and applying denoising to the gated images based on the noise analysis before motion estimation. This can generate denoised gated images with a balanced noise level (e.g., a same or substantially same noise level) across a whole set of denoised gated images.

For example, noise information associated with the gated images can be obtained through the noise analysis. A set of filter index values used to adapt or guide the denoising for the gated images can be determined based on the noise information, such that the entire set of denoised gated images achieves a balanced noise level. In this way, the denoised gated images input into the optical flow algorithm (or other motion estimation algorithms) have a more consistent noise level across different gates. This allows the motion estimation algorithm to more reliably identify corresponding textures and structures across the denoised gated images, thereby improving the accuracy of motion estimation.

In certain embodiments, noise analysis can be performed for each gated image individually with respect to a shared target noise level. Based on the analysis, a corresponding filter index value can be determined to indicate what degree of denoising should be applied to that gated image to reach the target noise level.

Alternatively, in other embodiments, noise analysis can be performed with respect to the entire set of gated images (e.g., relative to each other) to obtain an appropriate balanced noise level for that image set. Once the balanced noise level is obtained for the entire set, a respective filter index value can be determined for each gated image, so that denoising of the gated images generates denoised gated images at the balanced noise level.

In this way, the filter index value can serve as a denoising control parameter for each gated image. Therefore, the denoising can be adapted on an image-by-image basis, rather than applied uniformly across all gated images. As a result, the denoised gated images provided to the motion estimation algorithm can exhibit a balanced or substantially identical noise level.

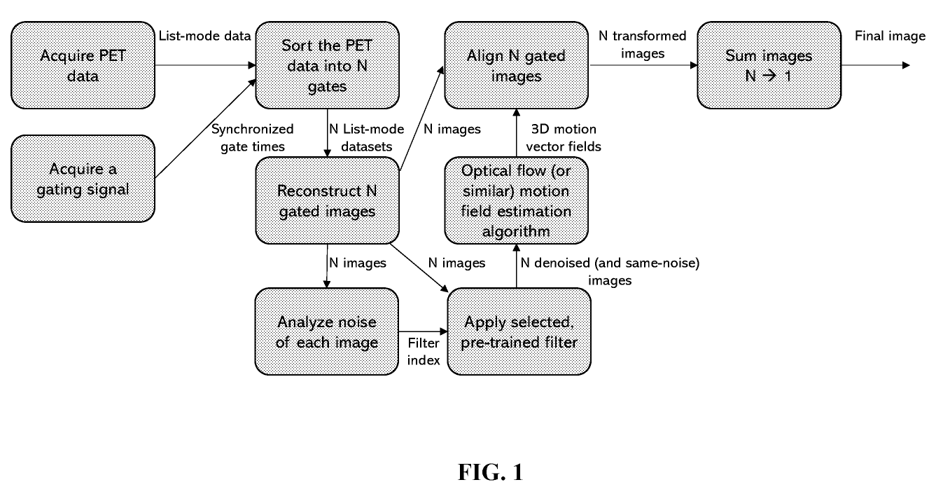

FIG. 1 shows an exemplary workflow of motion estimation and motion correction in accordance with embodiments of this disclosure. As shown, during PET acquisition, emission data can be acquired as list-mode data. Simultaneously, a gating signal can be acquired to provide synchronized gate times for the list-mode PET data.

Based on the gating signal, the acquired PET data can be sorted into a number (N) of gates, thereby deriving N list-mode datasets corresponding to the N gates. N gated images can be reconstructed from the N list-mode datasets. A noise analysis can be performed for the N gated images. Based on the analysis, N filter index values can be determined for respective gated images according to noise characteristics of the N gated images. These filter index values can be used to guide denoising for the N gated images.

Then, the generated filter index values are provided to a denoising stage. For example, the denoising stage can apply a selected denoising filter to each gated image based on the corresponding filter index value. With the control of the filter index values, the resulting N denoised images can achieve a consistent noise level across the N gates.

The N denoised images are provided to a motion field estimation stage. The motion field estimation stage can be an optical flow algorithm or a similar motion field estimation algorithm. From the N denoised images, the motion field estimation algorithm can estimate a three-dimensional (3D) motion vector field for each of the N gates relative to a reference gate. For example, since the end-expiration gate inherently shows the least amount of respiratory motion, it can be used as the reference gate.

Accordingly, the estimated 3D motion vector fields can be provided to an alignment stage to warp the N gated images to generate N transformed images. Each of the N transformed images corresponds to the motion state in the reference gate, e.g., a common position within a breathing cycle.

A motion compensated image can be generated based on the N transformed images, as a final image. For example, the final image can be generated by summing the N transformed images into a single image. Alternatively, the final image can be generated by averaging the N transformed images.

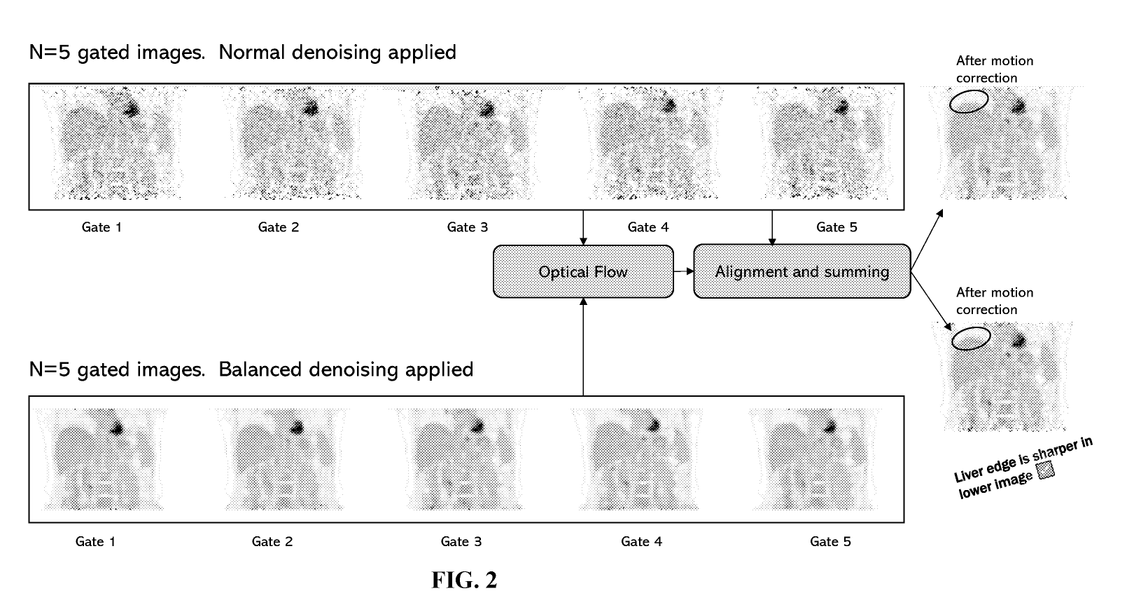

FIG. 2 illustrates a visual comparison using N=5 gated PET images to show the effects of different denoising schemes before motion estimation. In the upper portion, a normal denoising operation (e.g., linear smoothing) is applied to the gated images in a uniform or “blind” manner. The denoised images are then provided to an optical-flow-based motion estimation process, followed by alignment and summation to generate a motion-corrected image.

In the lower portion of FIG. 2, balanced denoising based on noise analysis is applied to the gated images before they are provided to the optical-flow-based motion estimation. In particular, the denoising applied to each gated image is adapted based on noise information obtained from analysis of the gated images, such that the denoised gated images have a more consistent noise level across the gates.

The final motion-corrected images generated from the two workflows are shown on the right side of FIG. 2. As shown, compared to the final image generated from the “blind” denoising workflow, the final image generated from the balanced-noise denoising workflow exhibits improved image quality, including a sharper edge of the liver.

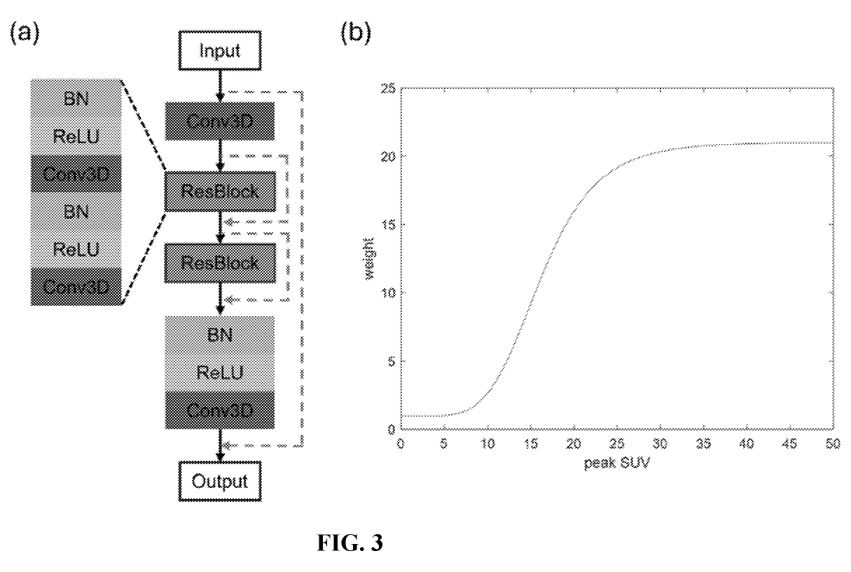

FIG. 3 illustrates an exemplary denoising deep convolutional neural network (DCNN) that can be used in the denoising stage in accordance with embodiments of the disclosure. As shown in part (a), the denoising DCNN includes an input layer, 3D convolution (Conv3D) layers, residual block (ResBlock) layers, batch normalization (BN) layers, rectified linear unit activation (ReLU) layers, and an output layer. Part (a) also schematically shows an exemplary internal structure of the ResBlock and exemplary residual connections within the DCNN.

Part (b) shows an exemplary weighting scheme for a weighted loss function that can be used to train the denoising DCNN. A loss weight can vary as a function of peak standardized uptake value (SUV), so as to weigh different SUV ranges during the training of the DCNN. The weighted loss function can be specifically designed to enhance the attention of the DCNN toward a certain SUV range, so as to place more emphasis on an image region that is more relevant to the final image reconstruction. For instance, an SUV range of 5 to 20 can be selected to prevent the DCNN learning being driven by high radiotracer intensities in the urinary system or large low-intensity background regions.

Additionally or alternatively, SUV clipping can be applied to increase the DCNN’s attention to selected SUV ranges and/or image regions.

Note that the illustrated network structure and weighting scheme are provided as an example only. Other DCNN architectures and/or other weighting schemes can also be used to implement the approaches provided in the disclosure.

By way of example, the denoising DCNN described above can be trained using a supervised learning process. The training dataset can include image data representing a broad range of imaging conditions, such as different gate numbers, different noise levels, different injected doses, etc., so that the DCNN can be trained with various images that may be encountered during the prediction (inference) phase. In certain embodiments, the denoising DCNN can be trained using a patch-by-patch training strategy. The network can be trained to minimize the weighted loss function using a gradient-based optimization algorithm, for example.

In an exemplary training process, full-count list-mode PET data can be randomly down-sampled to generate low-count data corresponding to lower count levels. Low-count images reconstructed from the low-count data can be used as noisy training inputs for the denoising DCNN. Images reconstructed from the full-count data can be post-smoothed using a Gaussian filter and used as training ground-truth images for the denoising DCNN.

For example, the full-count data can be list-mode PET data acquired over 10 minutes, and the low-count data can be generated by randomly down-sampling the full-count data to correspond to frame durations of 30, 45, 60, 90, 120, and 180 seconds.

In one embodiment, a plurality of neural networks can be trained, where each neural network is trained to handle a specific noise level (or a specific noise level range). During the inference phase, based on the noise analysis described above, corresponding filter index values can be determined for respective gated images according to noise characteristics of the gated images. Each filter index value can be used to select a trained neural network for a gated image. For example, the selected trained neural network can be one associated with a noise level that is closest to the filter index value determined for that gated image, or one associated with a noise level range that includes the determined filter index value.

In another embodiment, a single neural network is trained to handle various noise levels. In such implementations, the neural network can be trained to generate denoised outputs from inputs having different noise characteristics, such that the denoised outputs have a balanced noise level. During inference, each gated image can be input into the trained neural network together with a corresponding filter index value determined from the noise analysis described above. Thus, the denoising operation can still be adapted on an image-by-image basis using a single network model.

Although the above examples describe determining the filter index values based on the noise analysis of the gated images, the disclosed approaches are not limited thereto. One skilled in the art will appreciate that the filter index values can also be determined based on other information indicative of noise characteristics of the gated images.

For example, the filter index values can be determined based on one or more imaging parameters correlated with noise, including, but not limited to, gated frame duration (frame length), injected dose, patient body size (e.g., BMI), etc. In such implementations, the noise characteristics of the gated images can be estimated from the acquisition and/or reconstruction parameters, rather than determined from an image-based noise analysis.

For instance, a first frame duration can correspond to a first filter index value, a second frame duration can correspond to a second filter index value, and so on. Accordingly, the filter index values can be determined directly from the frame durations, and denoising can be adjusted based on the determined filter index values.

More generally, the filter index values described herein can represent denoising control parameters used to determine how denoising is performed for individual gated images. For example, when denoising is performed using a group of filters, the filter index values can indicate corresponding filters to be used for the gated images. As another example, when denoising is performed using a group of trained neural networks, the filter index values can be associated with corresponding neural network configurations and/or settings used in network training.

For instance, in a case where a plurality of trained neural networks are used, a first trained neural network can be trained using first ground-truth images generated by post-processing full-count images with a first Gaussian filter, a second trained neural network can be trained using second ground-truth images generated by post-processing full-count images with a second Gaussian filter, and a third trained neural network can be trained using third ground-truth images generated by post-processing full-count images with a third Gaussian filter. In this example, first, second, and third filter index values can be used to indicate selection of the first, second, and third trained neural networks, respectively.

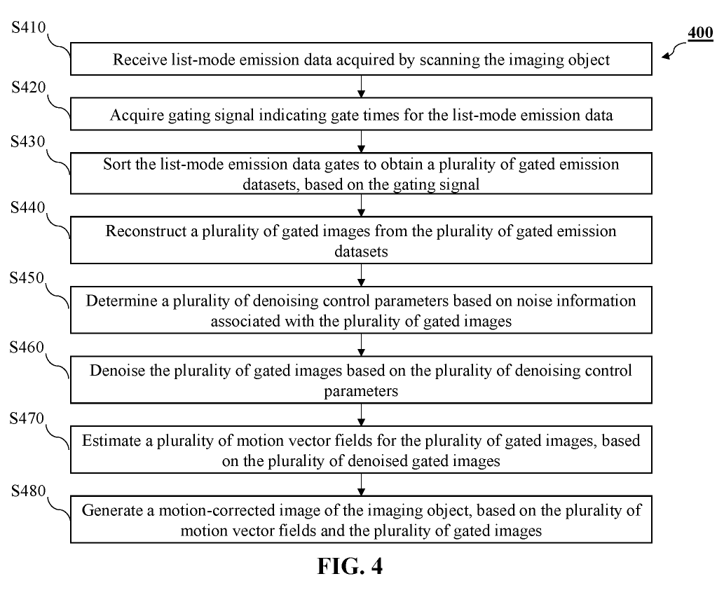

FIG. 4 shows a flow chart of an exemplary procedure for performing motion estimation and motion correction, in accordance with embodiments of the present disclosure.

As shown in FIG. 4, in step S410, list-mode emission data acquired by scanning an imaging object is received. In step S420, a gating signal can be acquired, where the gating signal provides gate times for the list-mode emission data. In step S430, based on the gating signal, the list-mode emission data can be sorted into a plurality of gates to obtain a plurality of gated emission datasets. In step S440, a plurality of gated images can be reconstructed from the plurality of gated emission datasets.

In step S450, a plurality of denoising control parameters can be determined based on noise information associated with the plurality of gated images. The noise information can be obtained in various ways, including by performing noise analysis with respect to the plurality of gated images and by estimating the noise information from one or more imaging parameters associated with the plurality of gated images.

In step S460, the plurality of gated images can be denoised based on the plurality of denoising control parameters, so as to generate a plurality of denoised gated images. Under the control of the plurality of denoising control parameters, noise levels among the plurality of denoised gated images can be balanced.

In step S470, a plurality of motion vector fields for the plurality of gated images can be estimated based on the plurality of denoised gated images. In step S480, a motion-corrected image of the imaging object can be generated based on the plurality of motion vector fields and the plurality of gated images.

Although the above examples are described with respect to PET imaging, the disclosed approaches can also be applied to single-photon emission computed tomography (SPECT) imaging. The motion to be estimated and corrected can include respiratory motion, cardiac motion, and other physiological motion.

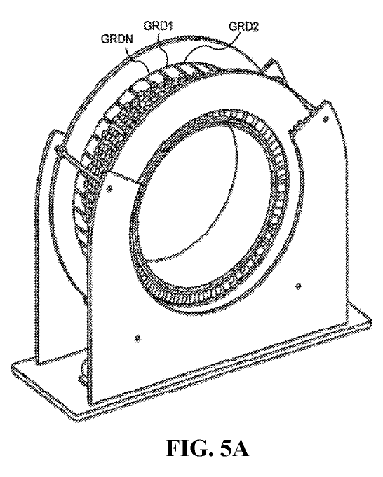

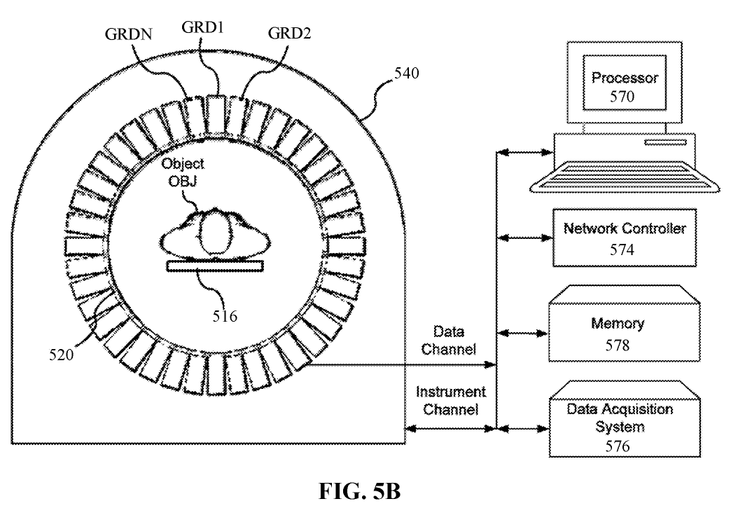

FIGS. 5A and 5B illustrate in implementation in which a medical imaging system includes a PET scanner that can implement the methods described in this disclosure. The PET scanner includes a plurality of gamma-ray detectors (GRDs) (e.g., GRD1, GRD2, through GRDN) that are each configured as rectangular detector modules.

Each GRD can include a two-dimensional array of individual detector crystals, which absorb gamma radiation and emit scintillation photons. The scintillation photons can be detected by a two-dimensional array of photomultiplier tubes (PMTs) or silicon photomultipliers (SiPMs). A light guide can be disposed between the array of detector crystals and the photodetectors.

Each photodetector (e.g., PMT or SiPM) can produce an analog signal that indicates when scintillation events occur, and an energy of the gamma ray producing the detection event. Moreover, the photons emitted from one detector crystal can be detected by more than one photodetector, and, based on the analog signal produced at each photodetector, the detector crystal corresponding to the detection event can be determined using Anger logic and crystal decoding, for example.

FIG. 5B shows one example of the arrangement of the PET scanner, in which the object OBJ to be imaged rests on a table 516 and the GRD modules GRD1 through GRDN are arranged circumferentially around the object OBJ and the table 516. The GRDs can be fixedly connected to a circular component 520 that is fixedly connected to a gantry 540. The gantry 540 houses many parts of the PET scanner. The gantry 540 of the PET scanner also includes an open aperture through which the object OBJ and the table 516 can pass, and gamma-rays emitted in opposite directions from the object OBJ due to an annihilation event can be detected by the GRDs and timing and energy information can be used to determine coincidences for gamma-ray pairs.

In FIG. 5B, circuitry and hardware are also shown for acquiring, storing, processing, and distributing gamma-ray detection data. The circuitry and hardware include: a processor 570, a network controller 574, a memory 578, and a data acquisition system (DAS) 576. The PET scanner also includes a data channel that routes detection measurement results from the GRDs to the DAS 576, the processor 570, the memory 578, and the network controller 574. The data acquisition system 576 can control the acquisition, digitization, and routing of the detection data from the detectors. In one implementation, the DAS 576 controls the movement of the bed 516. The processor 570 performs functions including reconstructing images from the detection data, pre-reconstruction processing of the detection data, and post-reconstruction processing of the image data, as discussed herein.

The processor 570 can be configured to perform various steps of the methods described herein and variations thereof. The processor 570 can include a CPU that can be implemented as discrete logic gates, as an Application Specific Integrated Circuit (ASIC), a Field Programmable Gate Array (FPGA) or other Complex Programmable Logic Device (CPLD). An FPGA or CPLD implementation may be coded in VHDL, Verilog, or any other hardware description language and the code may be stored in an electronic memory directly within the FPGA or CPLD, or as a separate electronic memory. Further, the memory may be non-volatile, such as ROM, EPROM, EEPROM or FLASH memory. The memory can also be volatile, such as static or dynamic RAM, and a processor, such as a microcontroller or microprocessor, may be provided to manage the electronic memory as well as the interaction between the FPGA or CPLD and the memory.

Alternatively, the CPU in the processor 570 can execute a computer program including a set of computer-readable instructions that perform various steps of the described methods, the program being stored in any of the above-described non-transitory electronic memories and/or a hard disk drive, CD, DVD, FLASH drive or any other known storage media. Further, the computer-readable instructions may be provided as a utility application, background daemon, or component of an operating system, or combination thereof, executing in conjunction with a processor, such as a Xeon processor from Intel of America or an Opteron processor from AMD of America and an operating system, such as Microsoft VISTA, UNIX, Solaris, LINUX, Apple, MAC-OS and other operating systems known to those skilled in the art. Further, CPU can be implemented as multiple processors cooperatively working in parallel to perform the instructions.

The memory 578 can be a hard disk drive, CD-ROM drive, DVD drive, FLASH drive, RAM, ROM or any other electronic storage known in the art.

The network controller 574, such as an Intel Ethernet PRO network interface card from Intel Corporation of America, can interface between the various parts of the PET scanner. Additionally, the network controller 574 can also interface with an external network. As can be appreciated, the external network can be a public network, such as the Internet, or a private network such as an LAN or WAN network, or any combination thereof and can also include PSTN or ISDN sub-networks. The external network can also be wired, such as an Ethernet network, or can be wireless such as a cellular network including EDGE, 3G and 4G wireless cellular systems. The wireless network can also be WiFi, Bluetooth, or any other wireless form of communication that is known.

Various techniques have been described as multiple discrete operations to assist in understanding the various embodiments. The order of description should not be construed as to imply that these operations are necessarily order dependent. Indeed, these operations need not be performed in the order of presentation. Operations described may be performed in a different order than the described embodiment. Various additional operations may be performed and/or described operations may be omitted in additional embodiments.

Embodiments of the present disclosure may also be as set forth in the following parentheticals.
(1) A method for performing motion estimation and correction in a medical imaging system, the method comprising: receiving list-mode emission data acquired by using the medical imaging system to scan an imaging object; acquiring a gating signal indicating gate times for the list-mode emission data; sorting, based on the gating signal, the list-mode emission data into a plurality of gates to obtain a plurality of gated emission datasets; reconstructing a plurality of gated images based on the plurality of gated emission datasets, wherein each gated image of the plurality of gated images is reconstructed from a respective one of the plurality of gated emission datasets; determining a plurality of denoising control parameters based on noise information associated with the plurality of gated images, wherein each denoising control parameter of the plurality of denoising control parameters corresponds to a respective one of the plurality of gated images; denoising, based on the plurality of denoising control parameters, the plurality of gated images to generate a plurality of denoised gated images, thereby balancing noise levels among the plurality of denoised gated images; estimating, based on the plurality of denoised gated images, a plurality of motion vector fields for the plurality of gated images, wherein each motion vector field of the plurality of motion vector fields corresponds to a respective one of the plurality of gated images; and generating a motion-corrected image of the imaging object, based on the plurality of motion vector fields and the plurality of gated images.
(2) The method of (1), wherein the step of determining the plurality of denoising control parameters further comprises: performing a noise analysis with respect to the plurality of gated images to obtain the noise information, and determining the plurality of denoising control parameters based on the obtained noise information.
(3) The method of (2), wherein the step of determining the plurality of denoising control parameters further comprises: for each gated image of the plurality of gated images, analyzing a noise level of the gated image with respect to a target noise level, and determining the denoising control parameter for the gated image, the denoising control parameter indicating a degree of denoising to be applied to the gated image to denoise the gated image to the target noise level.
(4) The method of (2), wherein the step of determining the plurality of denoising control parameters further comprises: analyzing the plurality of gated images to determine a common noise level for the plurality of gated images, and for each gated image of the plurality of gated images, determining the denoising control parameter for the gated image, the denoising control parameter indicating a degree of denoising to be applied to the gated image to denoise the gated image to the common noise level.
(5) The method of (1), wherein the step of determining the plurality of denoising control parameters further comprises: receiving one or more imaging parameters associated with the plurality of gated images, estimating the noise information based on the one or more imaging parameters, and determining the plurality of denoising control parameters based on the estimated noise information.
(6) The method of (5), wherein the one or more imaging parameters comprise: a gated frame duration, an injected dose, or a patient body size.
(7) The method of (1), wherein the step of denoising the plurality of gated images further comprises: for each gated image of the plurality of gated images, selecting, based on the denoising control parameter for the gated image, a trained neural network from a plurality of trained neural networks, and inputting the gated image into the selected trained neural network to generate a denoised gated image.
(8) The method of (1), wherein the step of denoising the plurality of gated images further comprises: for each gated image of the plurality of gated images, inputting the gated image and the denoising control parameter for the gated image into a single trained neural network to generate a denoised gated image.
(9) The method of (1), wherein the step of estimating the plurality of motion vector fields further comprises estimating the plurality of motion vector fields relative to a reference gate, and wherein the step of generating the motion-corrected image further comprises: warping the plurality of gated images based on the plurality of motion vector fields to obtain a plurality of warped images, and generating the motion-corrected image based on the plurality of warped images.
(10) The method of (1), wherein the medical imaging system is a positron emission tomography (PET) system or a single-photon emission computed tomography (SPECT) system.
(11) An apparatus for performing motion estimation and correction in a medical imaging system, the apparatus comprising: processing circuitry configured to receive list-mode emission data acquired by using the medical imaging system to scan an imaging object, acquire a gating signal indicating gate times for the list-mode emission data, sort, based on the gating signal, the list-mode emission data into a plurality of gates to obtain a plurality of gated emission datasets, reconstruct a plurality of gated images based on the plurality of gated emission datasets, wherein each gated image of the plurality of gated images is reconstructed from a respective one of the plurality of gated emission datasets, determine a plurality of denoising control parameters based on noise information associated with the plurality of gated images, wherein each denoising control parameter of the plurality of denoising control parameters corresponds to a respective one of the plurality of gated images, denoise, based on the plurality of denoising control parameters, the plurality of gated images to generate a plurality of denoised gated images, thereby balancing noise levels among the plurality of denoised gated images, estimate, based on the plurality of denoised gated images, a plurality of motion vector fields for the plurality of gated images, wherein each motion vector field of the plurality of motion vector fields corresponds to a respective one of the plurality of gated images, and generate a motion-corrected image of the imaging object, based on the plurality of motion vector fields and the plurality of gated images.
(12) The apparatus of (11), wherein the processing circuitry is further configured to determine the plurality of denoising control parameters by: performing a noise analysis with respect to the plurality of gated images to obtain the noise information, and determining the plurality of denoising control parameters based on the obtained noise information.
(13) The apparatus of (12), wherein the processing circuitry is further configured to determine the plurality of denoising control parameters by: for each gated image of the plurality of gated images, analyzing a noise level of the gated image with respect to a target noise level, and determining the denoising control parameter for the gated image, the denoising control parameter indicating a degree of denoising to be applied to the gated image to denoise the gated image to the target noise level.
(14) The apparatus of (12), wherein the processing circuitry is further configured to determine the plurality of denoising control parameters by: analyzing the plurality of gated images to determine a common noise level for the plurality of gated images, and for each gated image of the plurality of gated images, determining the denoising control parameter for the gated image, the denoising control parameter indicating a degree of denoising to be applied to the gated image to denoise the gated image to the common noise level.
(15) The apparatus of (11), wherein the processing circuitry is further configured to determine the plurality of denoising control parameters by: receiving one or more imaging parameters associated with the plurality of gated images, estimating the noise information based on the one or more imaging parameters, and determining the plurality of denoising control parameters based on the estimated noise information.
(16) The apparatus of (15), wherein the one or more imaging parameters comprise: a gated frame duration, an injected dose, or a patient body size.
(17) The apparatus of (11), wherein the processing circuitry is further configured to denoise the plurality of gated images by: for each gated image of the plurality of gated images, selecting, based on the denoising control parameter for the gated image, a trained neural network from a plurality of trained neural networks, and inputting the gated image into the selected trained neural network to generate a denoised gated image.
(18) The apparatus of (11), wherein the processing circuitry is further configured to denoise the plurality of gated images by: for each gated image of the plurality of gated images, inputting the gated image and the denoising control parameter for the gated image into a single trained neural network to generate a denoised gated image.
(19) The apparatus of (11), wherein the processing circuitry is further configured to estimate the plurality of motion vector fields by estimating the plurality of motion vector fields relative to a reference gate, and wherein the processing circuitry is further configured to generate the motion-corrected image by: warping the plurality of gated images based on the plurality of motion vector fields to obtain a plurality of warped images, and generating the motion-corrected image based on the plurality of warped images.
(20) The apparatus of (11), wherein the medical imaging system is a positron emission tomography (PET) system or a single-photon emission computed tomography (SPECT) system.

Those skilled in the art will also understand that there can be many variations made to the operations of the techniques explained above while still achieving the same objectives of the disclosure. Such variations are intended to be covered by the scope of this disclosure. As such, the foregoing descriptions of embodiments of the disclosure are not intended to be limiting. Rather, any limitations to embodiments of the disclosure are presented in the following claims.

What is claimed is:

1. A method for performing motion estimation and correction in a medical imaging system, the method comprising:
receiving list-mode emission data acquired by using the medical imaging system to scan an imaging object;
acquiring a gating signal indicating gate times for the list-mode emission data;
sorting, based on the gating signal, the list-mode emission data into a plurality of gates to obtain a plurality of gated emission datasets;
reconstructing a plurality of gated images based on the plurality of gated emission datasets, wherein each gated image of the plurality of gated images is reconstructed from a respective one of the plurality of gated emission datasets;
determining a plurality of denoising control parameters based on noise information associated with the plurality of gated images, wherein each denoising control parameter of the plurality of denoising control parameters corresponds to a respective one of the plurality of gated images;
denoising, based on the plurality of denoising control parameters, the plurality of gated images to generate a plurality of denoised gated images, thereby balancing noise levels among the plurality of denoised gated images;
estimating, based on the plurality of denoised gated images, a plurality of motion vector fields for the plurality of gated images, wherein each motion vector field of the plurality of motion vector fields corresponds to a respective one of the plurality of gated images; and
generating a motion-corrected image of the imaging object, based on the plurality of motion vector fields and the plurality of gated images.

2. The method of claim 1, wherein the step of determining the plurality of denoising control parameters further comprises:
performing a noise analysis with respect to the plurality of gated images to obtain the noise information, and
determining the plurality of denoising control parameters based on the obtained noise information.

3. The method of claim 2, wherein the step of determining the plurality of denoising control parameters further comprises:
for each gated image of the plurality of gated images,
analyzing a noise level of the gated image with respect to a target noise level, and
determining the denoising control parameter for the gated image, the denoising control parameter indicating a degree of denoising to be applied to the gated image to denoise the gated image to the target noise level.

4. The method of claim 2, wherein the step of determining the plurality of denoising control parameters further comprises:
analyzing the plurality of gated images to determine a common noise level for the plurality of gated images, and
for each gated image of the plurality of gated images, determining the denoising control parameter for the gated image, the denoising control parameter indicating a degree of denoising to be applied to the gated image to denoise the gated image to the common noise level.

5. The method of claim 1, wherein the step of determining the plurality of denoising control parameters further comprises:
receiving one or more imaging parameters associated with the plurality of gated images,
estimating the noise information based on the one or more imaging parameters, and
determining the plurality of denoising control parameters based on the estimated noise information.

6. The method of claim 5, wherein the one or more imaging parameters comprise:
a gated frame duration,
an injected dose, or
a patient body size.

7. The method of claim 1, wherein the step of denoising the plurality of gated images further comprises:
for each gated image of the plurality of gated images,
selecting, based on the denoising control parameter for the gated image, a trained neural network from a plurality of trained neural networks, and
inputting the gated image into the selected trained neural network to generate a denoised gated image.

8. The method of claim 1, wherein the step of denoising the plurality of gated images further comprises:
for each gated image of the plurality of gated images, inputting the gated image and the denoising control parameter for the gated image into a single trained neural network to generate a denoised gated image.

9. The method of claim 1, wherein the step of estimating the plurality of motion vector fields further comprises estimating the plurality of motion vector fields relative to a reference gate, and
wherein the step of generating the motion-corrected image further comprises:
warping the plurality of gated images based on the plurality of motion vector fields to obtain a plurality of warped images, and
generating the motion-corrected image based on the plurality of warped images.

10. The method of claim 1, wherein the medical imaging system is a positron emission tomography (PET) system or a single-photon emission computed tomography (SPECT) system.

11. An apparatus for performing motion estimation and correction in a medical imaging system, the apparatus comprising:
processing circuitry configured to
receive list-mode emission data acquired by using the medical imaging system to scan an imaging object,
acquire a gating signal indicating gate times for the list-mode emission data,
sort, based on the gating signal, the list-mode emission data into a plurality of gates to obtain a plurality of gated emission datasets,
reconstruct a plurality of gated images based on the plurality of gated emission datasets, wherein each gated image of the plurality of gated images is reconstructed from a respective one of the plurality of gated emission datasets,
determine a plurality of denoising control parameters based on noise information associated with the plurality of gated images, wherein each denoising control parameter of the plurality of denoising control parameters corresponds to a respective one of the plurality of gated images,
denoise, based on the plurality of denoising control parameters, the plurality of gated images to generate a plurality of denoised gated images, thereby balancing noise levels among the plurality of denoised gated images,
estimate, based on the plurality of denoised gated images, a plurality of motion vector fields for the plurality of gated images, wherein each motion vector field of the plurality of motion vector fields corresponds to a respective one of the plurality of gated images, and
generate a motion-corrected image of the imaging object, based on the plurality of motion vector fields and the plurality of gated images.

12. The apparatus of claim 11, wherein the processing circuitry is further configured to determine the plurality of denoising control parameters by:
performing a noise analysis with respect to the plurality of gated images to obtain the noise information, and
determining the plurality of denoising control parameters based on the obtained noise information.

13. The apparatus of claim 12, wherein the processing circuitry is further configured to determine the plurality of denoising control parameters by:
for each gated image of the plurality of gated images,
analyzing a noise level of the gated image with respect to a target noise level, and
determining the denoising control parameter for the gated image, the denoising control parameter indicating a degree of denoising to be applied to the gated image to denoise the gated image to the target noise level.

14. The apparatus of claim 12, wherein the processing circuitry is further configured to determine the plurality of denoising control parameters by:
analyzing the plurality of gated images to determine a common noise level for the plurality of gated images, and
for each gated image of the plurality of gated images, determining the denoising control parameter for the gated image, the denoising control parameter indicating a degree of denoising to be applied to the gated image to denoise the gated image to the common noise level.

15. The apparatus of claim 11, wherein the processing circuitry is further configured to determine the plurality of denoising control parameters by:
receiving one or more imaging parameters associated with the plurality of gated images,
estimating the noise information based on the one or more imaging parameters, and
determining the plurality of denoising control parameters based on the estimated noise information.

16. The apparatus of claim 15, wherein the one or more imaging parameters comprise:
a gated frame duration,
an injected dose, or
a patient body size.

17. The apparatus of claim 11, wherein the processing circuitry is further configured to denoise the plurality of gated images by:
for each gated image of the plurality of gated images,
selecting, based on the denoising control parameter for the gated image, a trained neural network from a plurality of trained neural networks, and
inputting the gated image into the selected trained neural network to generate a denoised gated image.

18. The apparatus of claim 11, wherein the processing circuitry is further configured to denoise the plurality of gated images by:
for each gated image of the plurality of gated images, inputting the gated image and the denoising control parameter for the gated image into a single trained neural network to generate a denoised gated image.

19. The apparatus of claim 11, wherein the processing circuitry is further configured to estimate the plurality of motion vector fields by estimating the plurality of motion vector fields relative to a reference gate, and
wherein the processing circuitry is further configured to generate the motion-corrected image by:
warping the plurality of gated images based on the plurality of motion vector fields to obtain a plurality of warped images, and
generating the motion-corrected image based on the plurality of warped images.

20. The apparatus of claim 11, wherein the medical imaging system is a positron emission tomography (PET) system or a single-photon emission computed tomography (SPECT) system.
